(19) United States
(12) Patent Application Publication
(10) Pub. No.: US 2025/0210150 A1
(43) Pub. Date: Jun. 26, 2025

(54) METHOD AND SYSTEM FOR OBTAINING CELL PERMEATION INFORMATION

(71) Applicant: University of Dundee, Dundee (GB)

(72) Inventors: Ulrich Zachariae, Dundee (GB); Dominik Gurvic, Dundee (GB)

(73) Assignee: University of Dundee, Dundee (GB)

(21) Appl. No.: 18/852,120

(22) PCT Filed: Mar. 27, 2023
(86) PCT No.: PCT/GB2023/050775

§ 371 Date: Sep. 27, 2024

(30) Foreign Application Priority Data
Mar. 28, 2022 (GB) 2204380.6

(51) Int. Cl. G16C 20/30; G16C 20/70

(52) U.S. Cl. CPC G16C 20/30; G16C 20/70

(57) ABSTRACT

A computer-implemented method of obtaining cell permeation information for one or more compounds, the method comprising: obtaining molecular structure data representative of molecular structure information for the one or more compounds; generating, using a pre-determined model, permeation data representative of cell permeation information for the one or more compounds based on at least the molecular structure data.

### FIELD

The present invention relates to a method and system for obtaining cell permeation information, for example, Gram-Negative permeation information.

### BACKGROUND

Over the past two decades, efforts have been made to derive general physicochemical composition rules to guide the design of new antibiotics by analysing existing drugs. Despite efforts including liquid chromatography-tandem mass spectrometry (LC-MS/MS) techniques, consensus regarding the key physical and chemical determinants of compound uptake for permeation has not yet been found.

The majority of severely drug-resistant bacterial infections are caused by Gram-negative (GN) bacteria, which account for two-thirds of the priority list of highly drug-resistant pathogens, published by the World Health Organisation (WHO) in 2017. Critical priority Gram-negative bacteria include Acinetobacte baumannii, Pseudomonas aeruginosa, Enterobacteriaceae, and Klebsiella pneumoniae. In the case of GN bacteria, bioactivity may be impeded by a high level of intrinsic resistance, arising from the poor drug permeability of the GN cell envelope. Overcoming the permeability barrier in the cell envelope of GN pathogens has been widely recognised as the key obstacle to the development of new broad-spectrum antibiotics, active against both Gram-positive (GP) and GN bacteria. It has been shown that the addition of terminal amine groups, among other factors, may improve the GN permeability of GP-active antibacterial compounds. However, adding terminal amine groups to a given lead compound may not always be feasible.

In recent years, Machine Learning (ML) approaches have been adopted in antibiotic discovery areas including lead generation, lead optimisation, and physicochemical property prediction. In “A Deep Learning Approach to Antibiotic Discovery” by Stokes et. al., a machine learning method was used to discover an active compound named Halicin.

### SUMMARY

In accordance with a first aspect, there is provided a computer-implemented method of obtaining cell permeation information for one or more compounds, the method comprising: obtaining molecular structure data representative of molecular structure information for the one or more compounds; generating, using a pre-determined model, permeation data representative of cell permeation information for the one or more compounds based on at least the molecular structure data.

The cell permeation information may comprise Gram-Negative permeation information. The permeation data may be representative of Gram-Negative permeation information. The method may further comprise storing the generated permeation data. The one or more compounds may comprise one or more antibiotic compounds. The generated permeation data may comprise or form part of a generated permeation data set for a further analysis. The method may further comprise performing at least one further processing step on the generated permeation data or generated permeation data set.

The method may further comprise processing at least the generated permeation data to determine one or more physical and/or chemical transformations and/or transformed compounds associated with a decrease or an increase in cell permeation.

The method may further comprise processing the cell permeation data to provide an estimate of antibacterial activity against a Gram-Negative bacteria for the one or more compounds.

The method may further comprise obtaining abstract confidence interval for the estimate.

The method comprises processing the cell permeation data to identify one or more drug candidates based on the cell permeation information. The method comprises processing the cell permeation data to identify one or more antibiotic candidates based on the cell permeation information.

The method may further comprise processing the generated permeation data to determine an experimental parameter for a subsequent drug validation process and/or to determine an operational parameter for an apparatus for drug validation and/or drug production and/or to determine a quantity or concentration of the compound that provides an increased level of antimicrobial activity.

The cell permeation information may be representative of at least one of: a degree of Gram-Negative permeation; a probability of Gram-Negative permeation; permeation of the one or more compounds into a Gram-Negative bacteria; permeability of a Gram-Negative bacteria to the one or more compounds; ability of the one or more compounds to cross a cell membrane or wall of a Gram-Negative bacteria.

The generated cell permeation data may comprise at least one score representative of cell permeation and wherein the method further comprises ranking and/or filtering the one or more compounds using said one or more scores.

The model may produce an effect on predicted Gram-Negative permeation for the one or more compounds from a change in one or more chemical and/or physical properties of the one or more compounds.

The one or more chemical and/or physical properties of the compound may comprise at least one of: a structural feature of the compound; a transformation of one or more structural features; one or more further chemical properties of the compound.

When applied to first input data the model may produce a first output and, when applied to second input data the model may produce a second output, such that the change in the first and second output is in response to changes in between the first and second input data. The change in the input data may correspond to a change in at least one structural feature; a transformation of one or more structural features; one or more further chemical properties of the compound.

The method may further comprise performing a further analysis using the obtained permeation data and the molecular structure data thereby to identify one or more molecular structural transformations that provide an increase in cell permeation and/or an increase in predicted cell permeation. The further analysis may comprise a matched molecular pair analysis,

The one or more transformations may correspond to a transformation from a GN-inactive compound to a GN-active compound.

Identifying the one or more molecular structural transformations may comprise identifying one or more differences in molecular structure between at least one pair of the plurality of compounds and determining a change in cell permeation associated with said one or more differences and/or wherein the analysis comprises performing a comparison of molecular connectivity information for one or more pairs of compounds.

The model may comprise at least one neural network, wherein the neural network is trained using a machine learning derived process. The pre-determined model comprise a predictive model. The pre-determined model may be trained using experimentally-obtained permeation data for a plurality of compounds. The method may comprise applying the predictive model to generate a synthetic permeation data set for a plurality of further compounds.

The pre-determined predictive model may comprise a machine learning model, for example, a neural network based model configured to be applied to one or more inputs to produce at least one output. The one or more inputs may correspond to at least said molecular structural information for the at least one compound. The at least one output may correspond to a predicted permeation value or other permeation information. The model may comprise an ensemble of trained models. Generating the permeation data may comprise applying the model to one or more inputs to produce an output. The one or more inputs may correspond to at least the structural information. The input data may further comprise one or more physical and/or chemical properties of the one or more compounds.

The molecular structure data may comprise a mathematical representation of the molecular structure, for example, a vector or matrix and/or one or more descriptive labels.

In accordance with a second aspect, which may be provided independently, there is provided a training method comprising: obtaining a permeation data set representative of at least cell permeation information for a plurality of compounds; obtaining a molecular structure data set representative of molecular structure information for the plurality of compounds; performing a model training process using at least the permeation data set and the molecular structure data set to train a model for generating at least cell permeation data from at least molecular structure data for one or more further compounds.

The molecular structure data for the one or more further compounds may be independent of the molecular structure data set. The permeation data set may comprise classification data. The classification data may comprise compounds classed as permeable and compounds classed as non-permeable. The generated permeation information comprises a prediction of cell permeation for the compound.

The method may comprise storing the trained model or data representative of the trained model. The model may be a machine learning derived model. The model may be a neural network model.

The one or more further compounds and the plurality of compounds may comprise at least one common structural feature and/or a common physical and/or chemical property.

The model training process may comprise determining an association between the molecular structure information and the cell permeation information for the plurality of compounds by processing the permeation data set and the molecular structure data set.

Training the model may comprise determining a relationship between cell permeation for the compound and one or more chemical and/or physical properties of the compound, wherein the one or more chemical and/or physical properties of the compound comprise at least one of: a structural feature of the compound; a transformation of one or more structural features; one or more further chemical properties of the compound.

Obtaining the permeation data set may comprise: obtaining antibacterial activity data representative of antibacterial activity for the plurality of antibacterial compounds against at least a Gram Negative bacteria; performing a classification process using the antibacterial activity data to classify the antibacterial compounds as at least one of:

Gram Negative permeable and Gram Negative impermeable; and obtaining Gram-Negative permeation information for the Gram Negative permeable antibacterial compounds. The training method may comprise performing a data curation process.

The antibacterial activity data may comprise minimal inhibitory concentration (MIC) values. The classification process may comprise applying a threshold function to the antibacterial activity data.

The method may further comprise processing a representation of the molecular structure to obtain descriptive textual data as a further input to the model.

The method may further comprise performing a validation of the trained model using in vitro permeation data.

The identified molecular transforms may comprise the addition and/or removal of at least one: Thiazole; ethylthiophene; Primary amine; Thiophenye; nitrile; Secondary amine; Ester; Lactone; Carbonyl; Carboxamide; Tertiary carboxamide; Aryl halide; Tertiary amine; Unsaturated carbonyl; Alkanol; Secondary carboxamide; Ether; Aniline.

In accordance with a third aspect, which may be provided independently, there is provided an apparatus comprising a processing resource configured to perform a method of obtaining cell permeation information for one or more compounds, the method comprising: obtaining molecular structure data representative of molecular structure information for the one or more compounds; generating, using a pre-determined model, permeation data representative of cell permeation information for the one or more compounds based on at least the molecular structure data.

The apparatus may comprises a first storage resource for storing the molecular structure data and a second storage resource for storing the permeation data.

In accordance with a fourth aspect which may be provided independently there is provided an apparatus comprising a processing resource configured to perform a training method comprising: obtaining a permeation data set representative of at least cell permeation information for a plurality of compounds; obtaining a molecular structure data set representative of molecular structure information for the plurality of compounds; performing a model training process using at least the permeation data set and the molecular structure data set to train a model for generating at least cell permeation data from at least molecular structure data for one or more further compounds.

The apparatus may comprises a first storage resource for storing the permeation data set and/or the molecular structure dataset and a second storage resource for storing the trained model.

In accordance with a fifth aspect, which may be provided independently, there is provided a computer program product comprising computer-readable instructions that are executable to perform the method of obtaining cell permeation information for one or more compounds, the method comprising: obtaining molecular structure data representative of molecular structure information for the one or more compounds;

generating, using a pre-determined model, permeation data representative of cell permeation information for the one or more compounds based on at least the molecular structure data.

In accordance with a sixth aspect, which may be provided independently, there is provided a computer program product comprising computer-readable instructions that are executable to perform a training method comprising: obtaining a permeation data set representative of at least cell permeation information for a plurality of compounds; obtaining a molecular structure data set representative of molecular structure information for the plurality of compounds; performing a model training process using at least the permeation data set and the molecular structure data set to train a model for generating at least cell permeation data from at least molecular structure data for one or more further compounds.

In accordance with a seventh aspect, there is provided a method comprising performing the computer-implemented method of the first aspect and further comprising the step of: selecting one or more of the compounds and applying at least one of the selected one or more compounds to a pathogen. The selecting of the one or more compounds may be based on the generated permeation data. The method may further comprise measuring the activity of the at least one applied compounds on the pathogen and/or the activity of a pathogen in the presence of the at least one compound.

Features in one aspect may be applied as features in any other aspect, in any appropriate combination. For example, features of the first aspect may be applied as features of the second aspect, or vice versa. Likewise, method features may be applied as features of an apparatus or a computer program product, and vice versa.

### BRIEF DESCRIPTION OF DRAWINGS

Various aspects of the invention will now be described by way of example only, and with reference to the accompanying drawings, of which:

FIG. 1 is a schematic diagram of a system configured to perform a method of generating cell permeation information, in accordance with embodiments;

FIG. 2 is a schematic diagram showing in overview a trained model for obtaining permeation information, in accordance with an embodiment;

FIG. 3 is a schematic diagram showing a workflow, including training and using the model of FIG. 2;

FIG. 4(a) is a table of permeation data for example compounds and FIG. 4(b) is a table of dataset parameters;

FIG. 5(a) is an illustration of a molecular transformation, FIG. 5(b) is a plot of permeation probability shifts after molecular transformations on a number of compounds and FIG. 5(c) is a plot of permeation probability difference due to transformations;

FIG. 6 is a plot of Gram Negative permeable and Gram Negative impermeable compounds in a parameter space;

FIG. 7 is a first table of results;

FIG. 8 is a second table of results;

FIG. 9 is a table illustrating molecular transformations and their respective changes in inhibition values, and

FIG. 10 is a third table of results.

### DETAILED DESCRIPTION

Consensus regarding the key physical and chemical determinants of compound uptake for bacteria, in particular, Gram-Negative bacteria has not yet been found. For Gram-Negative bacteria, it is thought that the cell envelope acts a strict permeation barrier to antimicrobial dugs. In the following a method of obtaining cell permeation information, for example, Gram-Negative permeation information, for one or more compounds is described.

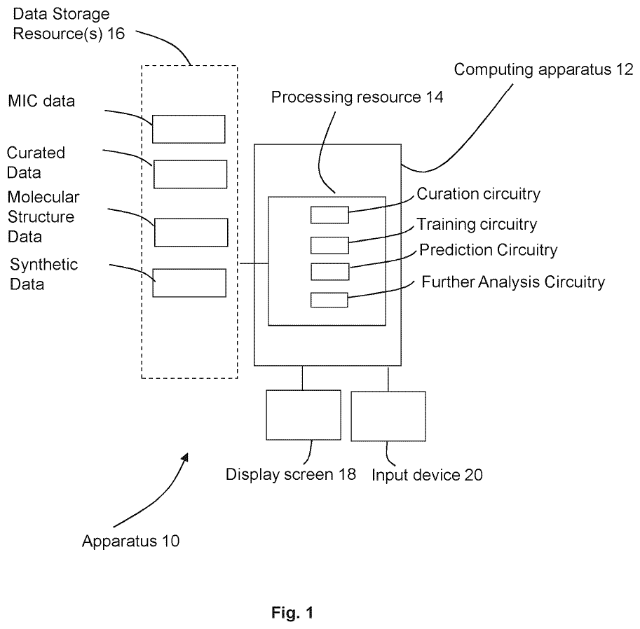

FIG. 1 is a schematic diagram depicting a computing apparatus 10 for performing a method of obtaining permeation information, in accordance with embodiments. FIG. 1 depicts a computing apparatus 12. The computing apparatus 12 has a processing resource 14, one or more data storage resources 16, a display screen 18 and an input device 20.

It will be understood that while FIG. 1 depicts a single computing apparatus for the purposes of the following description, the computing apparatus may be a distributed computing apparatus. For example, the computing apparatus 12 may comprise two or more computing apparatuses over a network. Likewise, the one or more data storage resources may be distributed data storage resources, for example, data may be retrieved from databases over a network.

The computing apparatus 12 comprises a processing resource 14. In the present embodiment, the processing resource 14 comprises a Central Processing Unit (CPU) and a graphic processing unit (GPU). For the purposes of the following description, the processing resource 14 has data curation circuitry, training circuitry, prediction circuitry and further analysis circuitry.

While each of these circuitries is depicted in the processing resource 14, it will be understood that in some embodiments, one or more of these circuitries are provided in circuitries of a further processing resource, for example, of a network connected computing resource. In particular, it will be understood that training may be performed on a further computing resource. In some embodiments, the training circuitry is implemented in the GPU. In the present embodiment, the curation circuitry, prediction circuitry and further analysis circuitry are implemented in the CPU. It will be understood that, in some embodiments, processing steps can be performed on a combination of computer processing units (CPUs) and graphics processing units (GPUs) and other dedicated processing units.

The circuitries may also be referred to, in some embodiments, as modules such that the apparatus has a curation module configured to curate data, training module configured to train a prediction model, prediction module configured to apply a prediction model and a further analysis module configured to perform one or more further analyses.

In the present embodiment, the various circuitries of the processing resource 14 are each implemented in the processing resource 14 by means of a computer program having computer-readable instructions that are executable to perform the method of the embodiment. However, in other embodiments each circuitry may be implemented in software, hardware or any suitable combination of hardware and software. In some embodiments, the various circuitries may be implemented as one or more ASICs (application specific integrated circuits) or FPGAs (field programmable gate arrays).

The computing apparatus 12 also includes a hard drive and other components including RAM, ROM, a data bus, an operating system including various device drivers, and hardware devices including a graphics card. Such components are not shown in FIG. 1 for clarity.

Turning to the data storage resources 16, for the purposes of the description, FIG. 1 depicts the data storage resources 16 having a number of different storage modules. The data storage resource 16 may include a hard drive or other suitable memory resource, for example, a network connected database. It will be understood that the data storage resources 16 may be a network distributed storage resource.

As described in the following, the data storage resources stores a number of different types of data including, for example, MIC data (also referred to as activity data), curated data, molecular structure data, synthetic data storage and permeation data storage. The data storage resources also provide a storage resource for trained model data. In particular, trained model parameters and model architecture parameters. In some embodiments, one or more of these types of data has a corresponding data storage resource and the processing resource is configured to perform the required data operations on the corresponding resource as required.

The display screen 18 may also be referred to as the display, for brevity. The input device 20 is configured to receive user input data representative of a user input. The user input and display may be considered to form a user interface.

While the circuitries are depicted in FIG. 1 as part of a single processing resource it will be understood that in some embodiments, one or more of the circuitries are provided as part of a further processing resource.

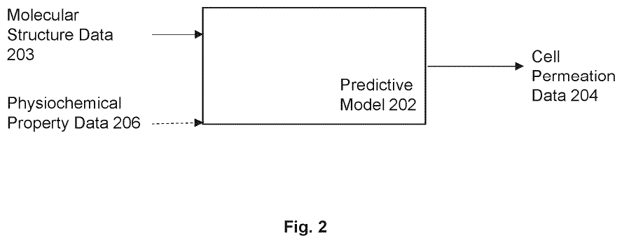

FIG. 2 is a schematic diagram depicting a predictive model 202 (referred to as simply a model for brevity) for obtaining cell permeation data 204 for a compound of interest, in accordance with embodiments. For a compound of interest, molecular structure data 203 is provided as input to the model 202. The molecular structure data 203 is representative of the molecular structure of the compound of interest and the model 203 relates the molecular structure information 203 to cell permeation data 204 for the compound. The molecular structure information 203 is provided to the model in the form of a mathematical representation of the molecular structure. In the present embodiment, the molecular structures are represented using the SMILES representation (Simplified molecular-input line-entry system) with binary labels. SMILES is a known representation for representing chemical structure using text strings. In some embodiments, obtaining the molecular structure data includes the steps of initially constructing a data structure and populating the data structure using structural data obtained from a data store. The structural data may be supplemented using physiochemical information.

In the present embodiment and as described in further detail in the following, the model is applied to the molecular structure data to output cell permeation data that is representative of cell permeation information. In the present embodiment, the permeation information is in the form of a predicted probability of Gram-Negative permeation. The predicted probability output is a value in the range 0 to 1. While, the above-described model outputs a prediction of probability for permeation, additional or alternative permeation information may be output by the model. The permeation information may correspond to, for example, an alternative measure corresponding to a degree of Gram-Negative permeation or the permeability of a Gram-Negative bacteria to the one or more compounds. The permeation information may represent, for example, an ability of the one or more compounds to cross a cell membrane or wall of a Gram-Negative bacteria. The permeation labels ‘0’ and ‘1’ were used for curated compounds, and the output predicted probability of Gram-Negative permeation is a probability between 0 to 1.

In some embodiments, applying the model includes the step of applying a model to the data structure to generate a further data structure representative of the permeation information. Generating the further data structure may include, constructing the further data structure for a compound, applying the model to the data structure to populate the further data structure with values representative of permeation information for the compound. In some embodiments, applying the model comprising applying an ensemble of more than one trained models.

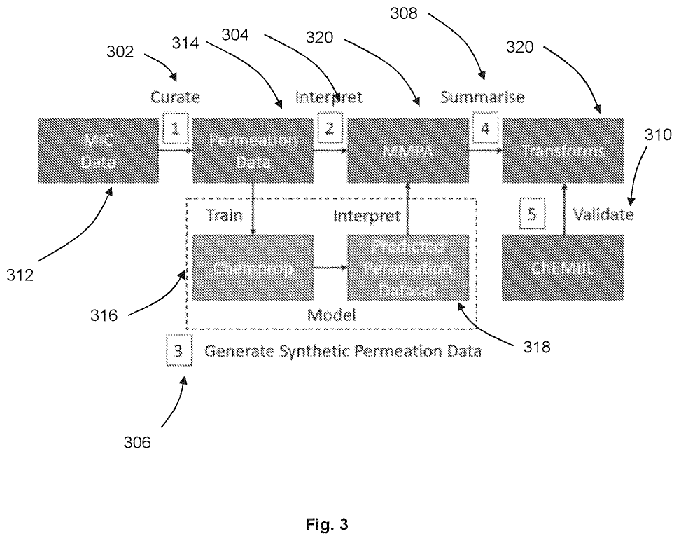

The model of FIG. 2 may be used in a number of different data processing workflows in which further data processing is performed on the output of the model. An example workflow is described with reference to FIG. 3. The further data processing may include obtaining physical and/or chemical transformations associated with the compounds that may provide an increased permeation probability or related metric. The further processing may include combining the obtained permeation information for more than one compounds and performing ranking, filtering or other selections of the compounds. The further processing may include determining one or more, or a subset of the compounds that are drug and/or antibiotic drug candidates based on the permeation information.

In the present embodiment, the input molecular structure data 203 is supplemented with additional physiochemical property data 206 relating to physical and/or chemical properties of the compound. In particular, in the present embodiment, physicochemical descriptors are provided as input to the model 202. The physiochemical descriptors are calculated by the chemoinformatic software package rdkit. Rdkit is an open source toolkit for cheminformatics. It will be understood that, while FIG. 2 depicts a model that receives supplementary physiochemical information associated with the compounds, in some embodiments, the model may be trained and used without such supplementary input.

In the present embodiment, the model is a neural network based model. As a non-limiting overview, such a neural network has a number of layers including a first layer (input layer), one or more hidden layers and an output layer. Each layer has one or more nodes. The connections between nodes of one layer and nodes each have an associated weight. The trained neural network weights therefore relate the input layer and the output layer. As the training or learning process is performed, the weights are iteratively adjusted. The neural network model may be considered as a series of equation that are produced using neural network modelling to allow prediction of permeation information using molecular structure data. The training is performed over time to refine the weights. In some embodiments, the input layer is a set of vectors of other data structure representing the molecular structure data and the method includes the step of constructing the set of vectors for use in the model. In some embodiments, the model is a series of equations that allow a prediction of permeation based on at least the molecular structural information and in such embodiments, the training of the mode may include producing the series of equations and the application of the model includes applying the equations. In some embodiments, the output layer is permeation information, for example, a score representative of permeation probability or a data structure that includes at least that permeation information or is representative of the permeation information.

As described in the following, in the present embodiment, the model is a neural network based model that is implemented using Chemprop software (MIT). In particular, the predictive model is an ensemble model of 5 neural networks.

FIG. 3 depicts a flow-chart illustrating, in overview, a workflow for training and using a model as described with reference to FIG. 2. The method of FIG. 3 can be considered to have five stages: data curation stage 302; interpretation stage 304; synthetic data generation stage 306; summarise stage 308 and a validation stage 310.

As an overview, publicly available minimal inhibitory concentration (MIC) data (also referred to as antibacterial activity data) was obtained and carefully curated to express it as data (curated data set) that is suitable to serve as proxy for GN permeation. An initial cycle of matched molecular pair analysis was performed and showed that, in this example, the initial curated dataset was too limited to provide a sufficient number of significant molecular structural changes (transforms) that would allow interpretation of the permeation data using MMP analysis alone. Therefore, machine learning techniques were used to train a model, as described with reference to FIG. 2, and to use the model to generate a large amount of additional synthetic data based on the initial curated dataset. The machine learning model was applied to the curated compounds of the curated data set to predict the probability of GN permeation for 2.6M new molecules thus forming synthetic data set(s). MMPA was then performed on 60,000 rationally selected molecules from the expanded synthetic dataset to identify transforms that have a substantial influence on permeability and emerge from a statistically significant number of structural modifications to molecular cores. Further analysis of the differing molecular structures in these transforms was performed to obtained major chemical determinants of permeation. Following independent validation of the resulting transforms by comparing them to data retrieved from the ChEMBL database, it was found that key determinants identified using the method may be suitable to serve as rules to guide molecular design for enhanced GN permeation

As depicted in FIG. 3, at the data curation stage 302, a data curation process was performed on antibacterial activity data (also referred to as activity data) to produce curated permeation data suitable for training a model. In the present embodiment, the antibacterial activity data corresponds to MIC (minimal inhibition concentration) data 312 for a number of compounds. The MIC data 312 included MIC values for the compounds against a number of GN and GP pathogens. The obtained curated data may also be referred to as permeation data 314. In the present embodiment, the data curation process is performed by processing resource 14, for example, curation circuitry.

As part of the data curation process, the MIC data 312 is retrieved from data storage resource(s) 16. In the present embodiment, the MIC data 312 is retrieved from remote databases, in particular: the Collaborative Drug Discovery (CDD) public database, which contains data on antibiotic activity from a variety of public and proprietary sources, as well as from the Community for Open Antimicrobial Drug Discovery (CO-ADD), an open access database which hosts screening results for compounds with potential antimicrobial activity.

In the present embodiment, the data curation process 302 includes the initial steps of data selection and filtering. The data selection and filtering steps may allow, for example, for a reduction of noise in the MIC data 312 and/or to allow removal of certain compounds that fail to meet threshold activity levels.

In further detail, it will be understood that the obtained MIC data 312 may be noisy. For example, in the present embodiment, the MIC data 312 includes data arising from different types of measurements and performed on different species. To reduce noise levels in data arising from different types of measurements and on different species, a data selection process is performed on the obtained MIC data 312, in which two paradigmatic pathogens are selected as representatives for each type of cell envelope. In the present embodiment, E. coli was selected as the GN bacteria and S. aureus as the GP bacteria. The selection of these two species allowed for the largest datasets for individual species, which at the same time originated from only a small range of different experimental procedures. The molecular targets for existing antibiotics inside the cells of S. aureus and E. coli are commonly thought to be homologous. This assumption is underpinned by the action of known broad-spectrum antibiotics that act on analogous targets across both GN and GP bacteria such as 3-lactams, quinolones, tetracyclins, and other antibiotic classes. In turn, this means that reduced activity levels of individual antibiotics within GN bacteria are likely to be caused by their diminished ability to permeate the GN cell envelope.

In the present embodiment, the data selection and filtering steps includes a compound filtering step in which compounds are filtered based on at least their activity level against one or more of the pathogens. In the present embodiment, only compounds that exhibit at least a medium-level activity against S. aureus and which have also been tested against E. coli (either as actives or non-actives) are retained to be labelled. In the present embodiment, the filtering step is performed following the data selection step, however, it will be understood that, in some embodiments, these steps are reversed.

The obtained MIC data includes a pMIC value against GN bacteria and a pMIC value against the GP bacteria. To curate the obtained data for use as training data, a classification process is performed on the obtained MIC data 312 to represent GN permeability as a binary classification. In the present embodiment, the classification process is performed on the selected and filtered data.

In the classification process, the compounds of the obtained data are initially split into two groups based on a comparison of their respective pMIC values for the GN bacteria and the GP bacteria to an activity threshold value (pMIC threshold value). In the present embodiment, pMIC is derived from the MIC values using the formula:

$$pMIC = - {log}_{10} ( MIC [ μ M ] \times 1 {0}^{- 6} )$$

An activity threshold was imposed at pMIC≤5 to focus on compounds with medium to high activity. Using this threshold, labels were assigned to all compounds, as follows.

A first label (‘1’) was assigned to compounds that can be considered to permeate both GN and GP cell walls and are active against both types of bacteria. Each compound assigned to this first group has a pMIC value against the selected GP bacteria (S. aureus) of greater or equal to the threshold value and a pMIC value against the selected GN bacteria (E. coli) of greater or equal to the threshold.

A second label (‘0’) was assigned to compounds that can be considered to permeate the GP cell wall but not the GN cell wall and show activity only on GP bacteria. Each compound assigned to this second group, has a pMIC value against the selected GP bacteria (S. aureus) of greater or equal to the threshold value and a pMIC value against the selected GN bacteria (E. coli) of less than the threshold.

The classification process allows a differentiating property to be created, in which the difference between compounds labelled as ‘0’ and ‘1’, and the barrier to activity against the selected GN bacteria (E. coli) is most likely caused by different permeation rates across the cell envelope (including both low inward uptake and active efflux).

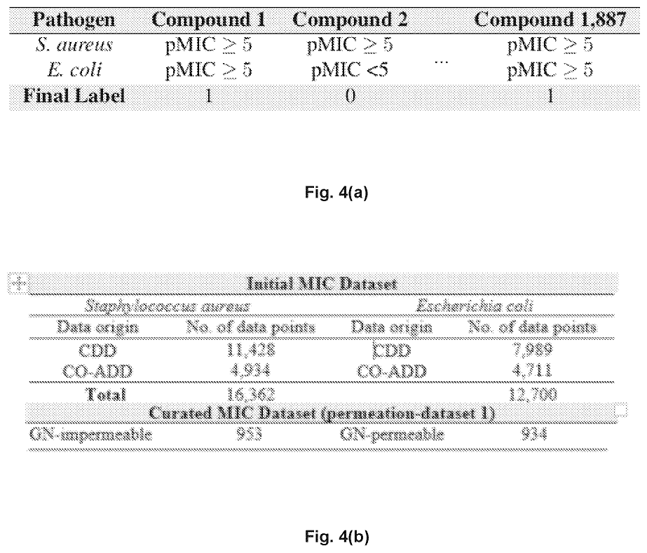

FIGS. 4(a) and 4(b) summarise the results of the selection, filtering and classification steps of the data curation process 302. Following the data curation process stage 302, the obtained permeation data 314 has 1,887 compounds, each compound classified as GN-permeable (assigned a value ‘1’) and GN-impermeable (assigned a value ‘0’). The classification provides a proxy for permeation. FIG. 4(a) shows three example compounds, their respective pMIC values and their assigned labels. As described above, compounds that are active against both selected pathogens above a threshold MIC value are labelled ‘1’ (GN-permeable), and compounds that are active against S. aureus, but inactive against E. coli are labelled ‘0’ (GN-impermeable).

FIG. 4(b) provides further data regarding the data curation step for the present embodiment, the initial MIC data was retrieved for 19,417 compounds from the CDD data, and further 9,645 MIC data points from CO-ADD data. These data were curated to represent GN permeation by applying the criteria described above. Applying these criteria led to 934 compounds labelled as ‘1’ (GN permeable or GN-active class) and 953 labelled as ‘0’ (GN impermeable or GN-inactive class). In the following permeation data 314 obtained via the data curation step is referred to as permeation dataset 1. For each compound, the permeation dataset has an obtained binary value (“1” or “0”) together with the structure information for each compound in SMILES representation. The compounds with pMIC labels come from the CDD/Co-ADD databases. Following curation, the curated dataset has fewer compounds with binary labels.

As discussed in further detail below, the permeation data 314 obtained by the data curation stage 302 is then interpreted (during the interpretation stage 304) by performing a further analysis (in this embodiment, a matched molecular pair analysis) on the permeation data. However, it was found that the matched pair analysis performed on the permeation dataset 1 did not pass a strict significance test. It has been found that publicly available bacterial permeation datasets may be too limited to be leveraged in a reliable way using such a matched molecular pair analysis.

Therefore, prior to performing this analysis, the workflow included a further stage of synthetic permeation data generation 306 to provide additional synthetic data for use in the interpretation stage. The process of generating synthetic data involved using machine learning techniques and training a model, such as the model described with reference to FIG. 2, to generate permeation information using at least molecular structure information. Alongside its use as a predictor for the GN permeability for any given new molecule, the trained model also allows for the synthetic expansion of the initial dataset, by predicting a learned property on large collections of new compounds. It may allow the underlying chemical features to be amplified and detected by further statistical analysis, even in the case where the original datasets are of limited size.

The training of the model was performed as follows. A first portion of the permeation data 314 obtained from step 302 was used as training data. A second portion of the permeation data 314 (in this embodiment, the portion is 15%) was set aside as test data. The test data is characterised by a balanced class distribution.

In the present embodiment, training and testing was performed using Chemprop (MIT) as indicated by reference 316. Before training the model, a built-in method for hyperparameter optimisation, which uses a Bayesian optimisation algorithm, was used on the training data. To optimise the prediction, an ensemble of five ML models was trained and a 5-fold cross-validation was performed. To promote wider generalisation of the ensemble, in the present embodiment, the molecular structural data were supplemented by additional physicochemical descriptors as calculated by the chemoinformatic software package rdkit. Further information for the software packaged rdkit may be found at http://rdkit.org/docs/Overview.html#what-is-it. In the present embodiment, both hyperparameter optimisation and training were carried out on GPU of processing resource 14. With reference to FIG. 1, the training can be considered to be performed by the training circuitry. It will be understood that, in some embodiments, the training circuitry may be a dedicated processing resource, for example, a distributed GPU cluster.

The Chemprop model uses a directed-message passing neural network to aggregate information from features of local atoms and bonds for every molecule in the training set, represented as a graph. In the present embodiment, the molecular structure information was combined with the associated activity/permeation label for each compound which was derived from the input datasets. The Chemprop model, after training, is capable of predicting the learned property, in this case, a prediction of cell permeation for new molecules that are not part of the initial training and test sets.

The initial hyperparameter optimisation was performed on the training dataset (85% of compounds of permeation dataset 1, with n=1604 compounds, 807 of which are GN permeable (or GN-active) and 797 are GN impermeable (or GN-inactive) resulted in the following parameters for all models used in the present study: hidden size of the neural network layers: 1700; number of message passing iterations: 6; dropout probability: 0.05; and number of feed-forward layers: 1. Using these parameters, a classifier consisting of an ensemble of five Chemprop models was trained and then 5-fold cross-validated, leading to a resulting overall training score of AUC=0.92±0.01. Finally, the ensemble was tested on the test set (the remaining 15% of compounds of the permeation data set, in which the number of compound, n was 283, of which 127 are GN permeable and 156 are GN impermeable) to achieve a test score of AUC=0.98.

The trained model obtained from the training process performed on the permeation data 314 was then applied on a number of independent external datasets to predict the permeation probability for a number of compounds. Each of the external datasets also include molecular structural data for a number of further compounds.

In the present embodiment, three external datasets (referred to as ENM_1, ENM_2 and ENM_3) were used. These datasets originated from the chemical synthesis company Enamine and together include molecular structure data for about 2.6 million compounds. The datasets ENM_1, ENM_2 and ENM_3 correspond to ‘FITS’, ‘Advanced’ and ‘Premium’, respectively, in the Enamine documentation. These datasets represent a wide range of physicochemical properties as well as a large variety of functional groups, thereby providing coverage of chemical space of the initial compounds.

The trained model was then used to generate synthetic data sets by processing the molecular structure data to generate predictions of permeation probability scores on a scale between 0 and 1 for each compound within three external datasets, ENM_1, ENM_2, ENM_3. The structures with labels (initial set) are used to train model to then use it on structures with no labels (ENM) to predict a label. Compounds in the ENM database are also represented using SMILES (for example, a compound is represented as: CC(C)Sc1nnc(-c2ccncc2)n1-c1ccc(F)cc1.

The synthetic permeation data is referred to as predicted permeation data 318 in FIG. 3. In the present embodiment, the generation of synthetic data was performed by processing resource 14, for example, by prediction circuitry.

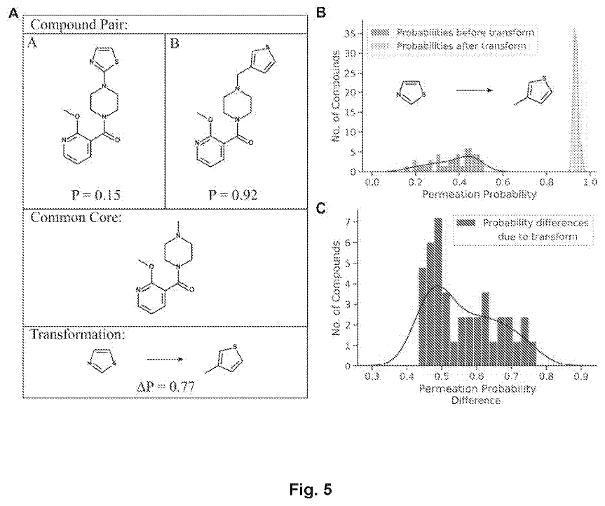

Matched Molecular Pair Analysis (MMPA) is a known method for comparing properties of pairs of molecules that differ only by a small structural change, known as a transformation. An example of a transformation is provided in FIG. 5. MMPA may be applied to large molecular datasets, generating a high number of pairs. MMPA can be used to compare multiple molecular properties at once, which makes MMPA a convenient multi-parameter optimisation tool. MMPA is used to analyse compounds with associated experimentally measured property or activity values. The MMPA is used with the probability scores as labels.

The interpretation stage 304 was then performed on the permeation data obtained from the synthetically generated datasets obtained from step 306. A matched pair analysis was applied to the datasets. Due to large computational costs associated with matched pair analysis, a pre-processing step was performed on the synthetic data to produce a truncated data set of pre-processed data. The truncation of the dataset is performed with the aim of maximising the molecular pairs in the pre-processed data and their score difference. With reference to FIG. 1, the matched pair analysis stage was performed using the further analysis circuitry of processing resource 14.

In further detail, similarity of the compounds was calculated (for example, by calculating a similarity score or other metric) by converting all compounds into extended connectivity fingerprints, where molecular structures are represented by bits in a binary vector. These representations of the molecular structures were then compared to each other by using a Jaccard-Tanimoto coefficient, which computes intersections of bits in the two binary vectors.

To optimise the selection of molecular pairs, i.e. compounds with a common core and therefore high molecular similarity, compounds within each dataset that exhibited no or low molecular similarity (at a 50% threshold) to the 10,000 highest-scoring compounds for permeability were discarded. From the remaining compounds (with a similarity above 50% to the top 10,000 compounds), the 10,000 molecules with the lowest permeability score were retained alongside the 10,000 high scorers. In this way, both the number of molecular pairs amongst the compounds in the pre-processed set and their score difference were maximised. This selection resulted in 20,000 compounds for each of the three ENM datasets, totaling 60,000 compounds. An MMPA 320 was then performed on the truncated data set. Each of the datasets, ENM_1, ENM_2, and ENM_3, containing compounds with predicted permeability scores, were analysed separately using MMPA.

The molecular transformations found through MMPA were then combined into a single dataset and subjected to statistical testing (paired t-tests), which yielded 2705 significant transforms. The average difference in permeation probability is 0.19, while the average number of repeats observed for each transformation is 7.75.

The chemical nature of the transforms was analysed to detect molecular substructures that consistently increase or decrease the predicted permeability throughout the whole dataset. For each transform, functional groups and moieties present in the left hand side (LHS) and right hand side (RHS) of each transform were identified separately. The LHS collection of substructures describe the lower permeability member of each transform and the RHS substructures characterise its higher permeability counterpart. The substructures were then compared to a predefined list consisting of 153 descriptors of functional groups and moieties in SMARTS notation.

The number of sub structural descriptors to those undergoing a significant change in the transformations by carrying out t-tests (p-value=0.01) on distributions prior to and after the transformation, followed by a Benjamini-Hochberg correction. Subsequently, there was a focus on transformations resulting in an increase in GN-activity, however, it will be understood that, due to the nature of the MMPA, every transform examined may be reversed to present statistically significant reductions in predicted activity. Descriptors representing generic substructures (for example, arene, heteroarene, alkyne, alkene nd azaarene) were discarded since they describe largely unspecific changes to the molecules encountered regularly in most transformations, which mostly do not contain useful information on feasible modifications. Taken together, these steps yielded 15 key descriptors that were retained for further analysis.

FIG. 5 depicts illustrative results using the further analysis performed on the synthetic permeation data. FIG. 5(a) depicts a compound pair A and B with a common core. The identified transformation leads to an increase in predicted GN permeation of 0.77. FIG. 5(b) is a graph depicted a permeation probability distribution before a transformation and a probability distribution after a transformation. The probability distribution is clearly shifted to higher values. The shift can be characterised by different distribution parameters, for example, a higher mean. The distribution also has a narrower spread (standard deviation). In this example, the shifted probability distribution does not overlap with the un-shifted distribution. FIG. 5(c) is a graph of difference in permeation probability difference due to transform.

At stage 308, the results from the MMPA were analysed and grouped into molecular transformations that substantially impact permeation.

As described above, a curated dataset (permeation dataset 1) was generated. Permeation dataset 1 may also be referred as GN-activity dataset 1. As described above, single-point MIC data for 19,417 compounds from the CDD, and further 9,645 MIC datapoints from CO-ADD were curated to form a proxy for GN permeation according to the criteria summarised in the table of FIG. 4(a). Applying these criteria led to 934 compounds labelled as ‘1’, GN-active class, and 953 labelled as ‘0’, GN-inactive class (GN-activity dataset 1).

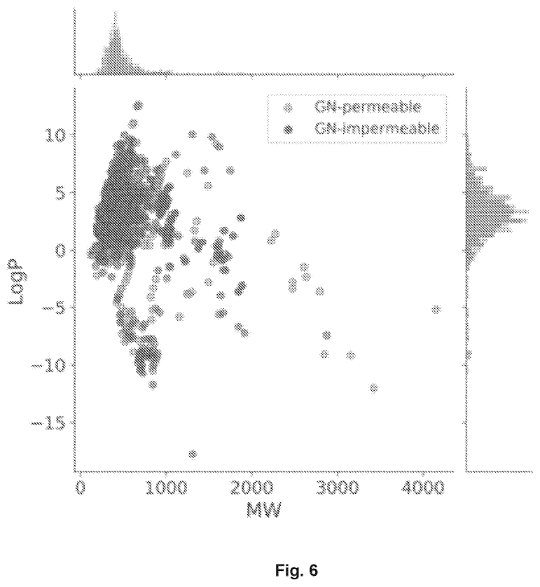

FIG. 6 is a plot of a chemical space occupied by GN-permeable (label 1) and GN-impermeable (label 0) compounds within permeation-dataset 1, represented by their molecular weight (MW) and hydrophobicity (log P) of the GN-activity dataset 1. The coverage of property space for the two distinct classes of molecules was studied. FIG. 6 shows the distribution of molecular weight (MW) and hydrophobicity (logP) for GN-active vs. inactive compounds. The GN-active molecules display a shift towards a smaller average log P value (l̅o̅g̅ P̅_{permeable}≈2.41, l̅o̅g̅ P̅_{impermeable}≈2.96), with the two distributions exhibiting a significant (p-value=1×10⁻⁴) difference to each other, although on the graph, the hydrophobicity regions occupied by the two types of molecules are largely inseparable. Furthermore, GN-active compounds exhibit a slight tendency towards larger average MW, (M̅W̅_{active}≈530.56, M̅W̅_{inactive}≈519.24), although no significant difference between the MW distributions is observed here (p-value=0.433).

Previous work, based on the analysis of less abundant compound datasets, has suggested that GN-permeable molecules tend to be more hydrophilic and smaller than GN-impermeable molecules. This effect has mainly been attributed to the selection criteria for permeating porin channels in the GN outer membrane. Porins possess a highly hydrophilic inner pore lining and a narrow, charged eyelet region which imposes an additional size, or MW, limitation on the spectrum of translocated molecules.

According to an analysis of 1,887 compounds, however, neither MW nor log P can serve as primary separator or predictor of activity or permeability across the GN cell envelope despite the small shift observed in logP. Recently, interactions between different classes of antibiotics and lipopolysaccharide in the GN bacterial outer membrane have been characterised, highlighting direct pathways into the outer membrane that do not involve porins. Similarly, it has been shown that permeating antimicrobials bypass the porins in the GN bacterial pathogen Pseudomonas aeruginosa. A greater diversity of inward permeation pathways than previously thought could, accordingly, explain the absence of clear MW or logP constraints on GN-activity in the present analysis and be responsible for the lack of consensus amongst previous studies.

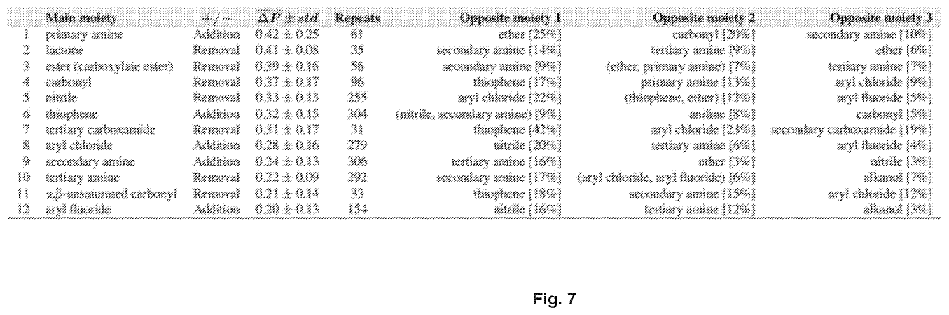

The table of FIG. 7 has generated key molecular transformations that are shown to significantly affect GN-activity scores of synthetically generated data. The table lists molecular replacements that correlate with a strong increase of the predicted GN-activity, which may be due to improved permeability according to our initial curation, either by addition or removal of each specific main moiety (+/−). The top three exchange counterparts in the matched molecular pairs are shown as opposite moieties. AP denotes the average change of the GN-activity score (limits from 0 to 1), linked to the respective transformations with associated standard deviation. Repeats shows the number of transformations which contain the ‘Main moiety’.

In further detail, sub-structural descriptors linked to a significant enhancement of the predicted GN-activity across the set of molecular transformations, as shown in the table of FIG. 7, were studied. The average increase within each subset (AP) was determined, whether addition or removal of the respective chemical moiety led to this increase (+/−), the number of transformations in which a particular moiety change was observed (Repeats), and the most commonly exchanged counter-moieties in the respective transformations (Opposite moieties, see below).

To illustrate our approach, the exemplar moiety, thiophene (Table of FIG. 7—row 6), was encountered in 304 out of the 2,705 transforms (Repeats). Taken together, these chemical trans-formations led to an average increase of 0.32 in the score. Here, this marked increase was observed for the addition of a thiophene moiety (+/−). In the transforms, thiophene most often replaced nitrile or secondary amine groups (joint opposite moiety 1, in 9% of the cases each), aniline (opposite moiety 2, in 8% of cases), and carbonyl groups (opposite moiety 3, in 5% of all cases).

The table of FIG. 7 shows that, on average, the addition of a primary amine group exerts the largest effect on increasing the activity score (Δ̅P̅≈0.42, represented by 61 transforms). The effect of primary amines is larger than that of secondary amines (Δ̅P̅≈0.24), represented in 306 transforms. These results are in excellent agreement with the previous findings. Such findings reported that primary amine groups increased GN permeability after examining a set of 180 chemically diverse compounds tested on E. coli. This underscores the validity of the approach based both on experimentally recorded and synthetic data, and using curated bioactivity data as a proxy for cell wall permeability.

A range of further moieties were shown to be associated with a substantial change in activity, similar to the level seen for primary amines (FIG. 7). For example, the removal of ester groups (56 repeats) and their cyclic subset, lactones (35 repeats), markedly improves the activity score (by Δ̅P̅≈0.41 and Δ̅P̅≈0.39, respectively).

While large, 14-16-membered lactone rings are known structural elements of the natural antimicrobial class, macrolides, the lactones examined in our transformations are mostly smaller rings with 3-6 members. The substitution of carbonyl (Δ̅P̅≈0.37), nitrile (Δ̅P̅≈0.33), and carboxam-ide groups (Δ̅P̅≈0.32) improves GN-activity by a similar extent. Notably, the addition of thiophene groups is only slightly less effective in increasing activity than the addition of a primary amine group (Δ̅P̅≈0.32). Furthermore, adding an aryl chloride group yields an activity increase of comparable magnitude (Δ̅P̅≈0.28), while the addition of secondary amines or aryl fluorides is also linked to a marked enhancement of GN-activity Δ̅P̅≈0.24 and Δ̅P̅≈0.20, respectively (FIG. 7).

While tertiary amines appear to be a second replacement choice for two moieties that are negatively correlated with improved GN-activity, on average they are themselves negatively correlated, especially when compared to primary & secondary amines, which suggests that replacing tertiary amines with secondary or primary amines increases the probability of permeation.

Overall, a clear pattern emerges of groups such as primary and secondary amines, thiophenes and aryl halides, which have large positive effects on GN-activity, especially when they replace substituent groups containing carbonyl oxygen (including esters, lactones, and carboxamides).

The experimental work thus demonstrates that, beyond the addition of primary amines and other nitrogen-containing groups, a range of alternative modifications to a given core molecule are likely to have similarly large effects on GN-activity.

Thus, in some embodiments the identified molecular transforms comprise the addition and/or removal of at least one of the group consisting of thiophene; thiazole; ethylthiophene (such as 2-ethylthiophene); primary amine (−NH₂); nitrile; secondary amine (such as —NH(C₁₋₆alkyl), e.g. —NHCH₃, —NHCH₂CH₃ or —NHCH(CH₃)₂); ester (such as —C(O)OR or —OC(O)R, where R is a hydrocarbyl, e.g. C₁₋₆alkyl); lactone; carbonyl; carboxamide (such as —C(ONR′R″ or —NR′C(O)R″, where R′ and R″ are each independently selected from H or hydrocarbyl, e.g. C₁₋₆alkyl); tertiary carboxamide (e.g. as carboxamide, wherein R′ and R″ are each independently hydrocarbyl); aryl halide; tertiary amine (such as —N(C₁₋₆alkyl)₂, e.g. —N(CH₃)₂, —N(CH₂CH₃)₂ or —N(CH(CH₃)₂)₂; unsaturated carbonyl; alkanol (such as —R″—OH, where R′″ is a bivalent hydrocarbon diradical, such as —CH₂— or —CH₂CH₂—); secondary carboxamide (e.g. as carboxamide, wherein one R′ or R″ is H and the other is hydrocarbyl); ether (such as —R^{a}OR_{b}, where R^{a} is a bivalent hydrocarbon diradical, such as —CH²— or —CH₂CH₂—, and R^{b} is a hydrocarbyl such as methyl, ethyl or phenyl); and aniline.

Although the predictive model was initially trained on rigorously curated measured MIC data, the statistical power of the MMPA performed relies on the use of additional synthetic data. It is important, therefore, to independently validate our results on datasets obtained exclusively from experimentally investigated compounds. In vitro data from the ChEMBL database was therefore screened for the presence of the patterns predicted to be linked to GN-activity and permeation.

The ChEMBL database merges MIC measurements obtained using a range of different assay types and from many different bacterial strains, including those in which bacterial perme-ation factors such as porins or drug efflux pumps were altered or deleted. The mixed composition of the ChEMBL dataset means that it may not be suited for use as training set as the more highly curated databases, CDD and Co-ADD; however, after a careful manual curation step, the data is arguably appropriate to serve as test set.

The validation process included: collecting all available inhibition data for both S. aureus and E. coli from ChEMBL, standardising all deposited inhibition units into pMIC, removing duplicated datapoints, and deleting datapoints resulting from assays involving mutated strains or strains with induced antibiotic susceptibility. This resulted in 24102 data-points for E. coli and 35802 for S. aureus (ChEMBL dataset 1).

Subsequently, this pMIC data was further curated to serve again as proxy for GN permeation data according to approach described above, yielding 5009 GN-active compounds (E. coli pMIC >5; S. aureus pMIC >5) and 2955 GN-inactive compounds (E. coli pMIC <5; S. aureus pMIC >5) (ChEMBL dataset 2).

To ascertain if the chemical transformations identified earlier increase the GN pMIC of a core molecule, MMPA was performed directly on the E. coli pMIC values (ChEMBL dataset 1). Separately, MMPA was carried out on the new permeation-proxy data to investigate if the transformations introduce additional GN-activity into GP-active molecules (ChEMBL dataset 2). The MMPA was followed by substructure search, matching any functional groups and moieties in the left hand side and right hand side of each transformation in both datasets to the previously identified activity-enhancing transforms that are likely due to improved permeability.

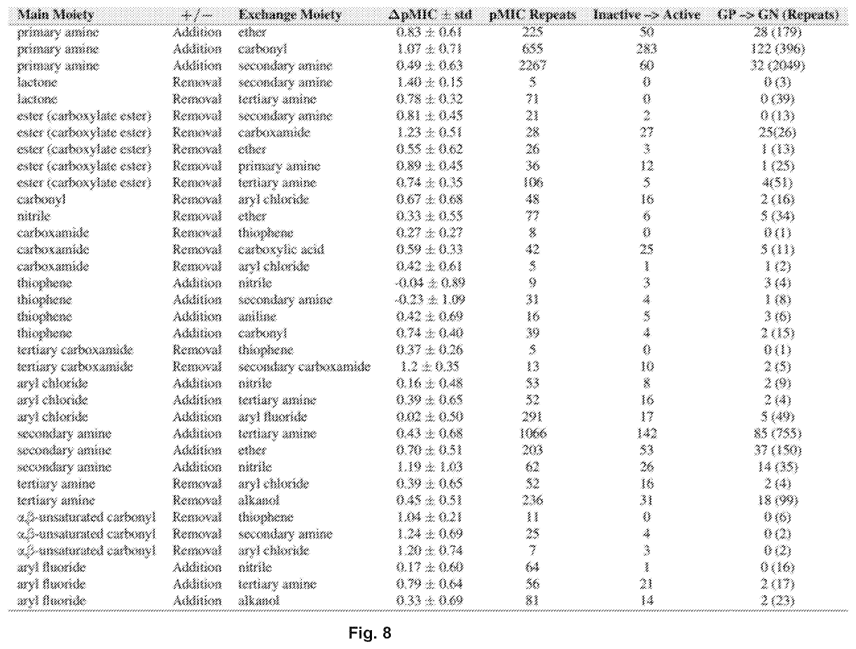

The Table of FIG. 8 shows validation of moiety exchanges predicted to improve GN-activity by screening in vitro E. coli MIC data and permeation-proxy data curated from ChEMBL. For every added or removed ‘Main moiety’ and its ‘Opposite moiety’ counterpart, as defined in FIG. 7, identical matched pairs were identified in the ChEMBL datasets. ‘pMIC Repeats’ denotes the number of identified pairs, ‘ΔpMIC’ denotes the average change in experimental pMIC, and ‘Inactive->Active’ the number of times the change in pMIC changed the core molecule from GN-inactive (pMIC <5) to GN-active (pMIC>5) in ChEMBL dataset 1. ‘GP->GN’ displays the number of times a core molecule is altered from GN-inactive to GN-active in the subset, ChEMBL dataset 2, with the number of repeats for the respective pairs in this dataset shown in parentheses

Furthermore, 86% (30/35) of the transformations turn at least one compound in the sets from GN-inactive to GN-active, and in 71% (25/35) of transforms in ChEMBL dataset 2, at least one example was found where the transform modifies a GP-only active compound into a compound that is active against both GP and GN bacteria.

The top row of the table of FIG. 8 shows 225 occurrences (‘pMIC Re-peats’) in ChEMBL dataset 1, where a primary amine is gained (‘Main Moiety’) in favour of an ether group (‘Ex-change Moiety’). These molecular transformations show an average positive increase in the E. coli pMIC of 0.83 (ApMIC). Furthermore, within those 225 examples, 50 cases in which this functional group substitution turns a GN-inactive compound into a GN-active molecule was found (according to the previously used definition, from activity below 5 pMIC to greater than 5 pMIC; ‘Inactive->Active’).

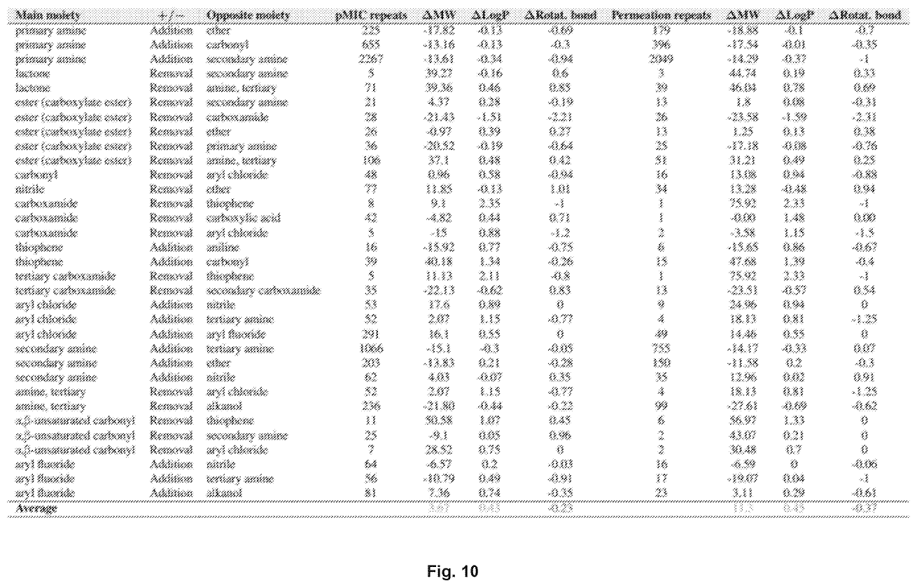

In the ChEMBL permeation-proxy dataset (ChEMBL dataset 2), 179 examples for the same molecular substitution were found. In 28 out of these cases, a GP-only active compound, by addition of primary amine in favour of an ether, is modified to become a broad spectrum active compound against both GP and GN bacteria (′GP->GN′). This independent screen of a large amount of experimental data provides further evidence that the molecular modifications suggested by our computational MMPA enhance GN-activity, and likely permeation in vitro. The vast majority of the transforms are found to have a substantially positive effect on the E. coli pMIC. Only two cases, of exchanging thiophene for nitrile or secondary amine, on average resulted in a negative pMIC change. A further five transforms failed to convert inactive compounds into active ones according to our pMIC definition (e.g. removal of lactone in favour of secondary amine). Further nine transforms did not convert compounds from Gram-positive active into Gram-negative active (e.g. removal of tertiary carboxamide in favour of thiophene), represented in the last two columns in the table of FIG. 10.

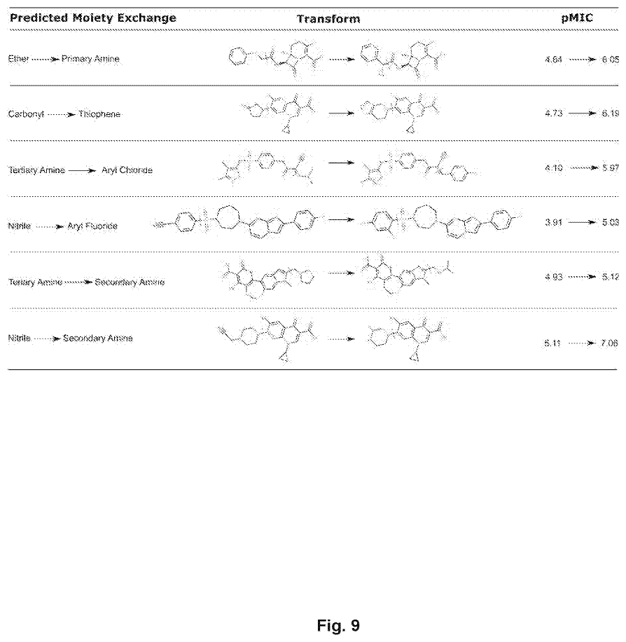

FIG. 9 shows six example transforms from the ChEMBL datasets where a detected significant fraction exchange leads to increased GN-activity. The dotted line indicates where the moiety alteration takes place, while the main molecular core remains identical. The first five examples turn a GN-inactive molecule into a GN-active compound according to our definition, while in the last example, a moderately GN-active molecule is modified into a highly GN-active one. (Note that in some of the cases the transforms contain more than only a change in the functional groups or moieties which were used to identify them, but our identified substructures are the common denominator, emerging from large and diverse datasets)

In further detail, FIG. 9 displays five examples of compound pairs retrieved from the ChEMBL database, in which the identified transforms improve GN-activity. In all five examples shown, GN-inactive compounds (pMIC <5) are rendered GN-active (pMIC >5) by the moiety exchange. In addition, a further exemplar transform, in which the molecular exchange turns a compound with borderline GN-activity into a molecule with high GN-activity is shown.

The table of FIG. 10 displays the change in molecular weight (MW), hydrophobicity (logP), and molecular flexibility (number of rotatable bonds) associated with the molecular substitutions that were identified to enhance GN-activity or permeation for ChEMBL datasets 1 (pMIC) and 2 (permeation). In further detail, the table of FIG. 8 displays the statistics obtained from screening the ChEMBL datasets for each of the main moieties and their exchange counterparts. As shown in the table, the ChEMBL in vitro data contains a large number of examples for the molecular substitutions that our previous computational analysis suggested to enhance GN-activity and permeation. It was found that 89% (31/35) of the computationally identified transforms indeed increase the E. coli in vitro pMIC in ChEMBL dataset 1. In two cases, no examples for our predicted exchanges were retrieved from the ChEMBL datasets (ether to lactone and aryl chloride to tertiary carboxamide).

Many previous analyses of GN-activity have investigated the physicochemical characteristics of compounds necessary to enable the crossing of the GN outer membrane, often focusing on their hydrophobicity (log P), molecular weight (MW) and rigidity of the structures. All of the chemical transformations that enhance GN permeation for systematic changes in these parameters were re-examined. The rigidity of a given molecule was assessed by determining its number of rotatable bonds.

The table of FIG. 8 shows that, while trends are observed for transformations that lead to the addition or removal of a specific group, trends between different types of transformations are usually dissimilar. For example, all transformations in which a primary amine is added reduce the hydrophobicity, whereas the addition of aryl chloride increases hydrophobicity. On average, only weak trends are seen overall, in which the log P is slightly raised along with MW, while molecules with improved GN-activity are slightly more rigid.

Taken together, these findings confirm that simple physico-chemical parameters are not well suited to differentiate between GN-active or permeable and non-permeable drugs due to their low degree of separation. According to the results, the presence or absence of specific chemical moieties, by contrast, serves as a much better predictor of GN-activity, and enables an interpretation of GN-activity or permeability on the basis of chemical properties. This is in agreement with recent meta-studies of GN compound uptake, where no consensus about the ideal physicochemical features optimising permeability has been reached.

The development of new broad-spectrum antibiotics with sufficient activity against both Gram-positive and Gram-negative pathogenic bacteria may be essential to address the drug-resistance problem emerging across a broad range of bacterial infections. Drug permeation across the Gram-negative cell envelope has been recognised as the primary obstacle in achieving a sufficient drug concentration and target activity in Gram-negative bacterial pathogens, and is a result of a complex interplay of multiple factors including outer membrane translocation and efflux. Although previous attempts to derive simple rules determining activity or permeation have had some success, there is, so far, no consensus amongst these studies regarding the roles of molecular features, which is likely primarily due to limitations in the amount of permeation data analysed.

In the absence of large intracellular drug concentration datasets, sizeable publicly available bacterial MIC datasets were used, rigorously curated to reduce noise from different experimental procedures and to optimally represent the effect of GN bacterial permeation. Machine learning was used to expand the available dataset by synthetically generating new compound-probability pairs from the known inhibition data. This dataset, containing 2.6M compounds in total, was then analysed for chemical features that influence GN-activity, by using Matched Molecular Pair Analysis. As described above, the results were validated by analysing available in vitro E. coli and S. aureus inhibition data from ChEMBL. The analysis highlights a number of molecular substructures that are consistently associated with enhanced GN-activity. These moieties include various amines, thiophenes, and halides, and thus potentially expand the medicinal chemistry toolbox beyond the previously suggested addition of terminal amine groups to enhance GN permeation. It was found that 86% of our predicted molecular modifications indeed improve E. coli growth inhibition in the independently analysed MIC data from ChEMBL. Furthermore, in 76% of the cases they promote GN bacterial permeation, according to our curated permeation proxy.

The analysis highlights a wide range of amine functions that improve GN-activity, which indicates that our computational model can successfully predict GN permeability and suggest experimentally validated molecular modifications to enhance up-take. Overall, a tendency of the more GN-active molecules to possess fewer rotatable bonds was found, i.e. a greater rigidity; however, the effect is moderate. A slightly positive correlation between the molecular hydrophobicity and GN-activity is observed in the study, which is in keeping with two previous LC-MS/MS studies that directly determined cellular accumulation. The approach therefore confirms several previous findings from experiments on compound permeation, but at the same time substantially widens the range of available modifications that can be made to a drug candidate to enhance its activity in GN bacteria.

The 2705 individual structural transforms that improve GN-activity provide specific examples of compounds and modifications that are optimising a given core structure for GN uptake. In order to aim for broader applicability, those transforms were analysed more deeply, in terms of recurring functional groups and moieties, to identify moiety exchange relationships (FIG. 8). These generalised moiety exchanges may serve as a resource for medicinal chemists to guide optimisation and synthesis of other core molecules in a more general way.

As described above, the trained model may be used in a number of different data processing workflows. In the above described workflow, the model was used to generate synthetic data including predicted permeation probabilities for use in a matched pair analysis. It will be understood that, the model may be used in a number of different workflows.

As a first non-limiting example, the output of the trained model may be used to predict physical or chemical transformations that lead to an improved permeation. In some embodiments, the output of the model itself may be a list of suggested transformations or transformed compounds that provide improved permeation.

In a further example, the model may receive structural data for a plurality of compounds and the output may include a ranked or filtered list of the compounds where the ranking/filtering is based on the output permeation score. In such a way, a subset of the input compounds can be reduced to indicate potential drug candidates. For example, the output permeation probability may be compared to a threshold value (for example, such a value could be 0.8) such that all compounds having a permeation probability output lower than the threshold value are discarded.

In a further example, the permeation information output by model may be processed to provide other parameters, for example, one or more experimental parameters for a subsequent drug validation process or an operational parameter for an apparatus for drug validation and/or drug production. For example, the permeation information output may be used to predict inhibitory concentration for a compound. As a further example, different amounts or concentrations of compounds may provide different levels of cell permeation such that an optimum amount or concentration provides optimum level of antimicrobial activity. In some embodiments, the model is trained to directly output such parameters.

In further embodiments, the model may be trained to output additional information together associated with the permeation information, for example, an error value associated with the generated permeation probability.

A skilled person will appreciate that variations of the enclosed arrangement are possible without departing from the invention. Accordingly, the above description of the specific embodiments are made by way of example only and not for the purposes of limitations. It will be clear to the skilled person that minor modifications may be made without significant changes to the operation described.

1. A computer-implemented method of obtaining cell permeation information for one or more compounds, the method comprising:
obtaining molecular structure data representative of molecular structure information for the one or more compounds;
generating, using a pre-determined model, permeation data representative of cell permeation information for the one or more compounds based on at least the molecular structure data.

2. The method according to claim 1, wherein the cell permeation information comprises Gram-Negative permeation information.

3. The method of claim 1 further comprising:
processing at least the generated permeation data to determine one or more physical and/or chemical transformations and/or transformed compounds associated with a decrease or increase in cell permeation.

4. The method according to claim 1, wherein the method comprises processing the cell permeation data to provide an estimate of antibacterial activity against a Gram-Negative bacteria for the one or more compounds.

5. The method according to claim 1, wherein the method comprises processing the cell permeation data to identify one or more antibiotic candidates based on the cell permeation information.

6. The method according to claim 2, wherein the cell permeation information is representative of at least one of: a degree of Gram-Negative permeation; a probability of Gram-Negative permeation; permeation of the one or more compounds into a Gram-Negative bacteria; permeability of a Gram-Negative bacteria to the one or more compounds; ability of the one or more compounds to cross a cell membrane or wall of a Gram-Negative bacteria.

7. The method according to claim 1, wherein the generated cell permeation data comprises at least one score representative of cell permeation and wherein the method further comprises ranking and/or filtering the one or more compounds using said at least one score.

8. The method according to claim 1, wherein the model produces an effect on predicted Gram-Negative permeation for the one or more compounds from a change in one or more chemical and/or physical properties of the one or more compounds.

9. The method according to claim 1, wherein the one or more chemical and/or physical properties of the compound comprise at least one of: a structural feature of the compound; a transformation of one or more structural features; one or more further chemical properties of the antibiotic compound.

10. The method according to claim 1 further comprising performing a further analysis, for example a matched molecular pair analysis, using the obtained permeation data and the molecular structure data thereby to identify one or more molecular structural transformations that provide an increase in cell permeation and/or an increase in predicted cell permeation.

11. The method according to claim 9, wherein the one or more transformations correspond to a transformation from a GN-inactive compound to a GN-active compound.

12. The method according to claim 10, wherein identifying the one or more molecular structural transformations comprises identifying one or more differences in molecular structure between at least one pair of the plurality of compounds and determining a change in cell permeation associated with said one or more differences and/or wherein the analysis comprises performing a comparison of molecular connectivity information for one or more pairs of compounds.

13. The method according to claim 1, wherein at least one of a), b):
a) the model comprises at least one neural network, wherein the neural network is trained using a machine learning derived process;
b) the molecular structure data comprises a mathematical representation of the molecular structure, for example, a vector or matrix and/or one or more descriptive labels.

14. (canceled)

15. A training method comprising:
obtaining a permeation data set representative of at least cell permeation information for a plurality of compounds;
obtaining a molecular structure data set representative of molecular structure information for the plurality of compounds;
performing a model training process using at least the permeation data set and the molecular structure data set to train a model for generating at least cell permeation data from at least molecular structure data for one or more further compounds.

16. The training method according to claim 15, wherein the one or more further compounds and the plurality of compounds comprise at least one common structural feature and/or a common physical and/or chemical property.

17. (canceled)

18. The training method according to claim 15, wherein at least one of a), b);
a) training the model comprises determining a relationship between cell permeation for the compound and one or more chemical and/or physical properties of the compound, wherein the one or more chemical and/or physical properties of the compound comprise at least one of: a structural feature of the compound; a transformation of one or more structural features; one or more further chemical properties of the compound;
b) the model training process comprises determining an association between the molecular structure information and the cell permeation information for the plurality of compounds by processing the permeation data set and the molecular structure data set.

19. The training method according to claim 15, wherein obtaining the permeation data set comprises:
obtaining antibacterial activity data representative of antibacterial activity for the plurality of antibacterial compounds against at least a Gram Negative bacteria;
performing a classification process using the antibacterial activity data to classify the antibacterial compounds as at least one of: Gram Negative permeable and Gram Negative impermeable; and
obtaining Gram-Negative permeation information for the Gram Negative permeable antibacterial compounds.

20. The method according to claim 19, wherein the antibacterial activity data comprises minimal inhibitory concentration (MIC) values and/or wherein the classification process comprises applying a threshold function to the antibacterial activity data.

21. The method according to claim 15, wherein at least one of a), b), c):
a) the method further comprises processing a representation of the molecular structure to obtain descriptive textual data as a further input to the model;
b) the method further comprises performing a validation of the trained model using in vitro permeation data;
c) the identified molecular transforms comprise the addition and/or removal of at least one: Thiazole; ethylthiophene; Primary amine; Thiophenye; nitrile; Secondary amine; Ester; Lactone; Carbonyl; Carboxamide; Tertiary carboxamide; Aryl halide; Tertiary amine; Unsaturated carbonyl; Alkanol; Secondary carboxamide; Ether; Aniline.

22. (canceled)

23. (canceled)

24. An apparatus comprising a processing resource configured to perform the method of claim 1.

25. (canceled)

26. An apparatus comprising a processing resource configured to perform the training method of claim 15.
